(19) United States
(12) Patent Application Publication
(10) Pub. No.: US 2012/0245662 A1
(43) Pub. Date: Sep. 27, 2012

(54) Wrap around cooling apparatus or assembly

(76) Inventor: George Page, Chicago, IL (US)

(21) Appl. No.: 13/070,718

(22) Filed: Mar. 24, 2011

(51) Int. Cl. A61F 7/00

(52) U.S. Cl. 607/112

(57) ABSTRACT

A compartmentalized wrap around cooling element, assembly, or device for cooling relief or swelling may be provided. The compartmentalized wrap may include a series of poly-vinyl alcohol dividers to provide distinctly separate cooling sections, for long lasting cooling relief. Tube connector joints, tube loops, button snaps, and Velcro connectors may provide a secure means for forming a wrap around or loop, to encircle an area multiple times. An encasement design may provide an alternative means of securing the wrap to a large area, such as an adult head.

### CROSS-REFERENCE TO RELATED APPLICATIONS

This application claims the benefit of U.S. Provisional Patent Application Ser. No. 61/315,921, filed on Mar. 20, 2010, entitled “Compartmentalized Wrap around Cooling Element”, which is expressly incorporated herein by reference in its entirety.

### COPYRIGHTS

All rights, including copyrights, in the material included herein are vested in and the property of the Applicant. The Applicant retains and reserves all rights in the material included herein, and grants permission to reproduce the material only in connection with reproduction of the granted patent and for no other purpose.

### BACKGROUND

According to the Centers for Disease Control, during 1999 and 2003, 3442 deaths occurred from exposure to extreme heat. Out of the 3442 deaths, 40% occurred in adults aged 65 or older. The human body has the ability to cool itself from perspiration. But, amongst athletes, heatstroke is the third leading cause of death. In many cases, perspiration alone is not enough to cool the body to acceptable core temperatures necessary to prevent serious injuries or death from excessive overheating. Physical activity in extremely hot temperatures is the leading cause of heatstroke.

Air conditioning is an accepted means of cooling a body temperature. Unfortunately, when air conditioning is unavailable, a portable cooling device must be used to cool the body. Cold packs are typically used to provide an instant cooling, as packs are able to actively reach temperatures close to ice within a few minutes.

In the case of injury, cold or ice packs are necessary to prevent swelling. Ankle sprain is the most common form of injury requiring a cold pack to reduce swelling. In cases where athletes have excessive use on joints or chronic injuries, ice packs are also effective in reducing swelling. Swelling may also result from; heat edemas, burns, bruising, and allergic reactions, among other causes.

A heat edema is the result of overwork in extreme heat conditions. In hot temperature conditions, blood vessels dilate. This dilation causes excessive body fluids to move to the hands or legs by gravity resulting in swelling. Burns can occur from the following exposure circumstances; electrical, chemical, sun, fire, and inhalation. When a burn occurs, the victims capillaries leak. A large amount of fluid leaks out of the tissue into the burn area, resulting in swelling.

When the skin suffers a blow, tiny blood vessels are damaged and broken causing leaking into the injured area thus, leaving a pool of blood under the skin. As a result, rising of the skin occurs, or bruising, also known as a contusion. Bruising typically turns the skin purple, due to blood accumulating under the skin, a condition often referred to as ecchymosis.

The immune system overreacts when exposed to certain chemicals causing mast cells and basophils with IgE on their surface to release histamine, prostaglandins, and leukotrienes that cause swelling in the surrounding tissues. The level of swelling depends on individual sensitivity to allergens. The resulting swelling varies from mild to severe.

When swelling occurs, ice is needed to reduce swelling and provide pain relief. In many cases, refrigeration is unavailable to keep ice. Thus, a cold source which does not require refrigeration is helpful in many circumstances. Ice packs currently on the market activate by a user breaking a pouch of water to mix with ammonium nitrate pellets, the result of which is a cooling mixture which reaches 36 degrees Fahrenheit.

U.S. Pat. No. 7,509,692 discusses a wearable personal cooling and hydration system with a pouch located within a backpack. Wearing a backpack adds significant weight to the user. Thus, this is not an ideal cooling method for exercising, such as running, where additional weight limits mobility.

U.S. Pat. No. 7,291,164 discusses an ICE Powerpack which provides cold therapy thru hollow vessels filled with water. This Powerpack requires initial refrigeration to freeze the device. Since refrigeration is not available in remote areas, the device is ineffective in remote situations where some injuries occur.

U.S. Pat. No. 5,557,807 discusses a headware including cooling means which cools by using alternative commercial packs within the unit. Commercial packs are designed to last for approximately 10 minutes and thus a single use will not provide lasting cooling to a user. Additionally, the cooling packs themselves are very bulky and need further design in order to provide functional use within a hat. Conventional cooling elements are in the form of cold packs, which effectively cool to approximately 36 degrees Fahrenheit, but a large pack weighs approximately 1 pound. The weight of the pack renders the pack ineffective to use during physical activity. During exercise or fitness activity, cold packs are ineffective.

Cold packs often can cool up to a maximum of 15 minutes. This relatively short cooling period render a single cold pack inadequate in most situations when swelling occurs. When additional cooling is necessary, multiple cold packs are needed. This can result in carrying multiple pounds of cooling packs, creating bulk to transport, and excessive waste upon discarding.

What is needed is a portable, light-weight, and inexpensive cooling device which effectively cools various body parts when physical activity is required in excessive heat or the head needs to be cooled quickly.

### SUMMARY

This summary is provided to introduce a selection of concepts in a simplified form that are further described below in the Detailed Description. This summary is not intended to be used to limit the claimed subject matter's scope.

Conventional devices or elements which cool a human body are bulky and inefficient in providing a lasting cooling source. Embodiments of the invention address the need for a compact, lightweight, long lasting cooling device or assembly which allows for cooling multiple parts of the body to address various health needs. Since the embodiments may not contain costly mechanisms, they may be extremely cost effective for a consumer.

Embodiments of the invention may provide a cooling element or assembly that reaches adequate cooling to 36 degrees and can maintain that temperature for 1 or more hours, and is lightweight for transportability. Embodiments of the invention include a compartmentalized wrap around cooling device, assembly, or element that provides long lasting cooling by compartmentalizing, or segmenting cooling into a series of sections. This is done thru incorporated polyvinyl alcohol divider segments soluble at temperatures for example above 50-60 degrees. The first segment is activated by contact of water with ammonium nitrate pellet contents, to activate cooling of the first segment. After approximately 15-30 minutes for instance, the segments cooling reaction will lessen by the first segment temperature rising, for example, to 60 degrees, from higher body temperature absorption. This will cause the poly-vinyl alcohol divider segment to solubilize to permit the 60 degree liquid to contact the ammonium nitrate from segment 2, thus activating cooling in segment 2. The process continues for as many segments that exist. Each segment may create, for example, approximately 30 minutes of cooling, thus multiple segments can allow hours of cooling to occur for a single user.

Through a wrap-around design, an area is encased or surrounded, eliminating the need to have one larger surface area cold pack to cover an effected area. The flexibility of the compartmentalized wrap around cooling element or device allows it to circle multiple times over the same area. Thus, a specific area may cool in parallel, encircling an area to allow segments to align next to one another, increasing the number of times an area is cooled.

Both the foregoing general description and the following detailed description provide examples and are explanatory only. Accordingly, the foregoing general description and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present invention. In the drawings:

FIG. 1 shows a perspective view of a cold wrap or wrap around cooling element;

FIGS. 2A-2C shows side views of connector assemblies and the view of a cold wrap with the connector assemblies in place;

FIG. 3 shows alternative connectors for a cold wrap;

FIG. 4 shows an encasement design for the cold wrap; and

FIG. 5 shows an exploded cross section perspective view of an open cold wrap.

### DETAILED DESCRIPTION

The following detailed description refers to the accompanying drawings. Wherever possible, the same reference numbers are used in the drawings and the following description to refer to the same or similar elements. While embodiments of the invention may be described, modifications, adaptations, or modifications may be made to the elements illustrated in the drawings, and the methods described herein may be modified by substituting, reordering, or adding stages to the disclosed methods. Accordingly, the following detailed description does not limit the invention.

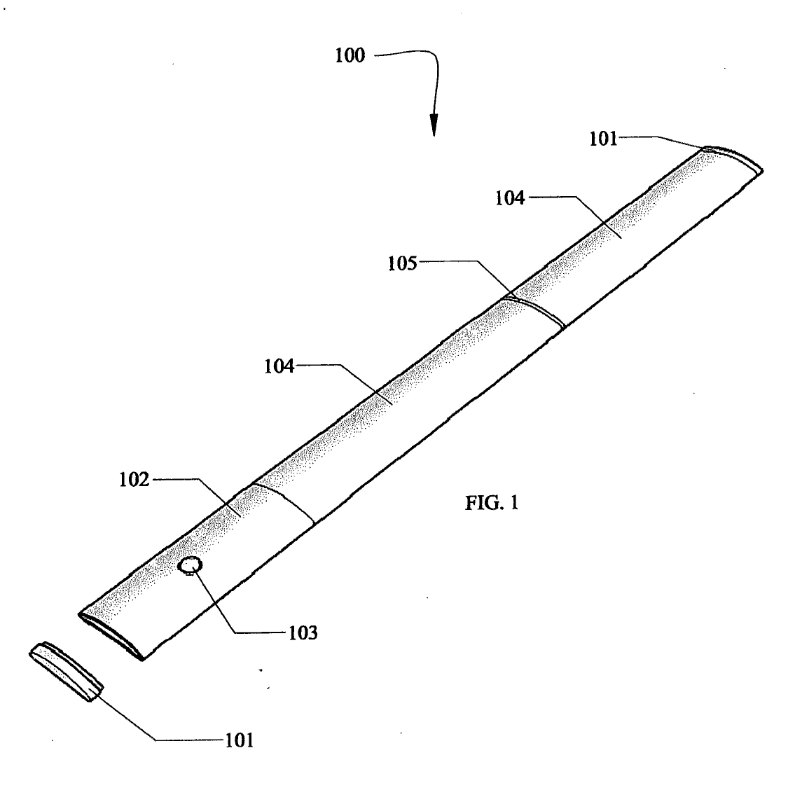

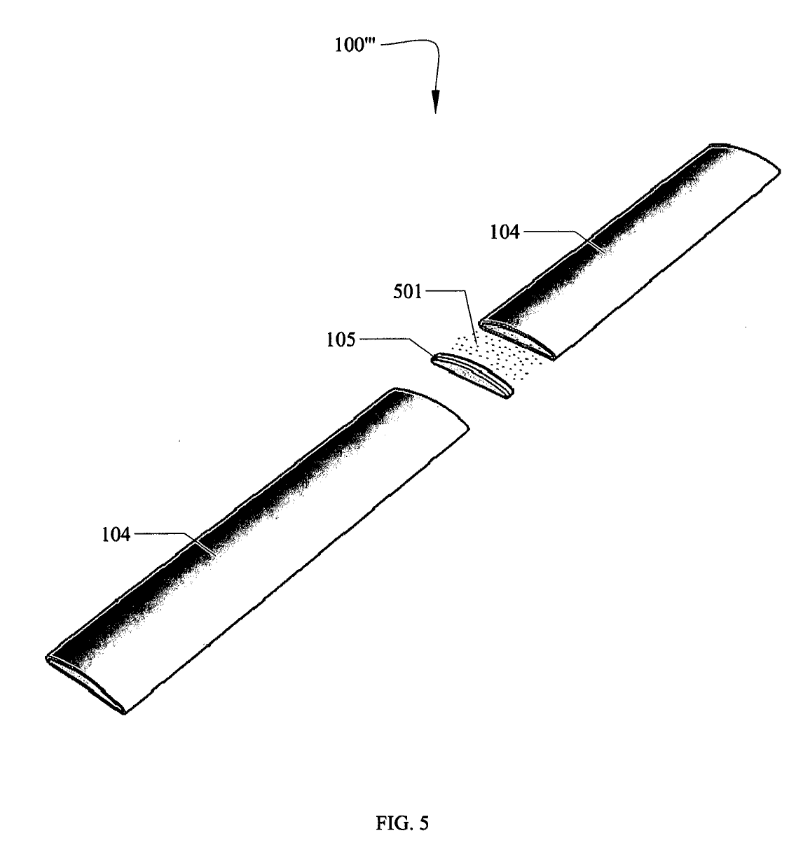

FIG. 1 shows a perspective view of a flexible cold wrap 100, possibly made of plastic or silicone, including compartments or a wraparound cooling element 100. The cold wrap 100 may provide cooling relief by removing end cap 101 possibly made of silicone or plastic, which once removed allows access to activate cooling, and applying pressure to the release button 103 that includes an activating mechanism, possibly a puncture element for example, a sharp pin, to release the contents of water pouch 102. When the contents of water pouch 102 are placed into direct contact with a cooling material such as ammonium nitrate pellets 501 (see FIG. 5) contained in a first compartment 104, the first compartment 104 is lowered to a temperature of approximately 36 degrees Fahrenheit (“F”). Upon completion of the cooling, for instance represented by the first compartment 104 reaching a temperature of 60 degrees F., a solubilizing poly-vinyl alcohol divider 105 allows the liquid contents of the first compartment 104 to travel into a next or second compartment 104. Upon interaction of ammonium nitrate pellets 501 with the contents of the first compartment 104, the next compartment 104 begins to cool to a temperature of approximately 36 degrees F. The process continues until all compartments 104 have similarly reacted.

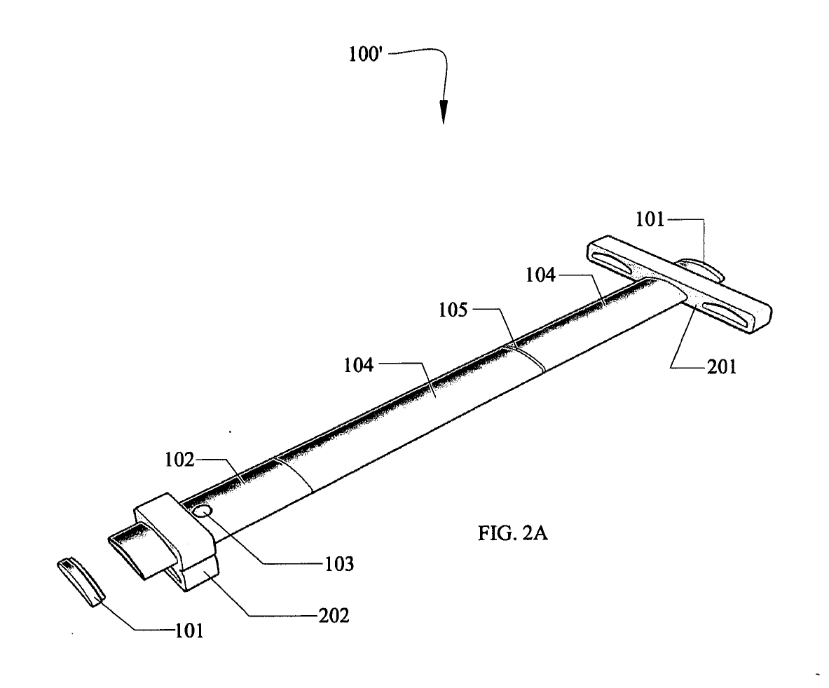

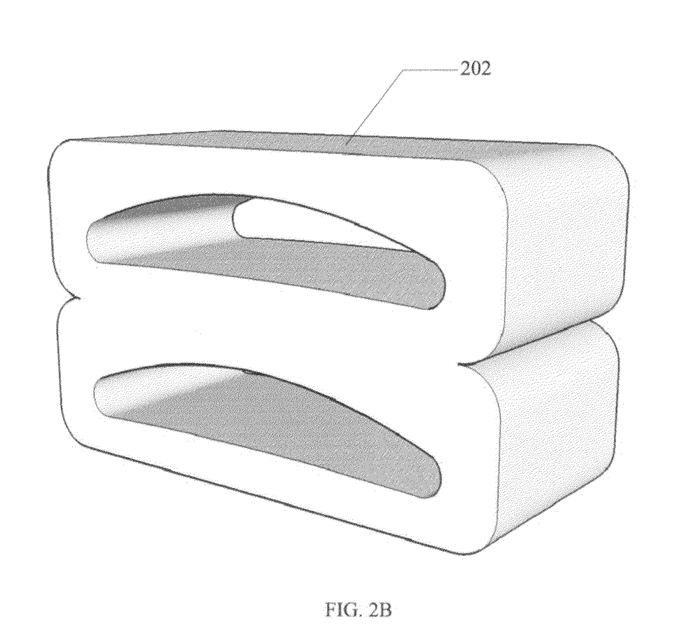

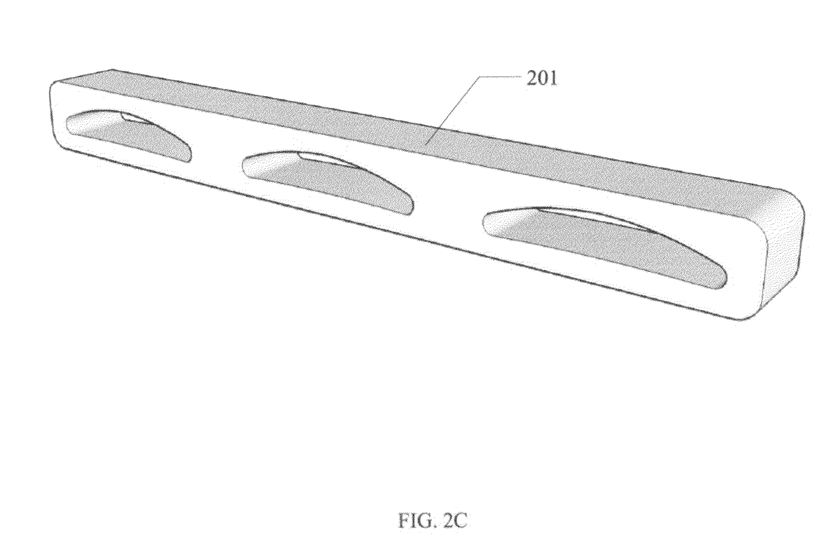

FIGS. 2A-2C shows side views of connector assemblies 201 and 202 and a perspective (substitute) view of a compartmentalized cold wrap 100′ with the connector assemblies 201/202 in place. The cold wrap 100′ may encircle an affected area of a human body to provide additional contact surface area of a specific region of the body. The cold wrap 100′ may extend, for example, up to 18 inches allowing for various configurations. When the coverage area is less than 8 inches, such as the cross section circumference of a wrist, ankle, neck, leg or arm, the cold wrap 100′ may loop twice into tube connector assembly 201 joints, to permit parallel cooling. Parallel cooling allows for compartments to align one next to another to increase coverage of a specified area.

When the cooling area cross section circumference exceeds 18 inches, such as; an adult head, back or stomach, the cold wrap 100′ may only loop once. To secure a tight loop, connector assembly 202 secures both ends of the cold wrap 100′.

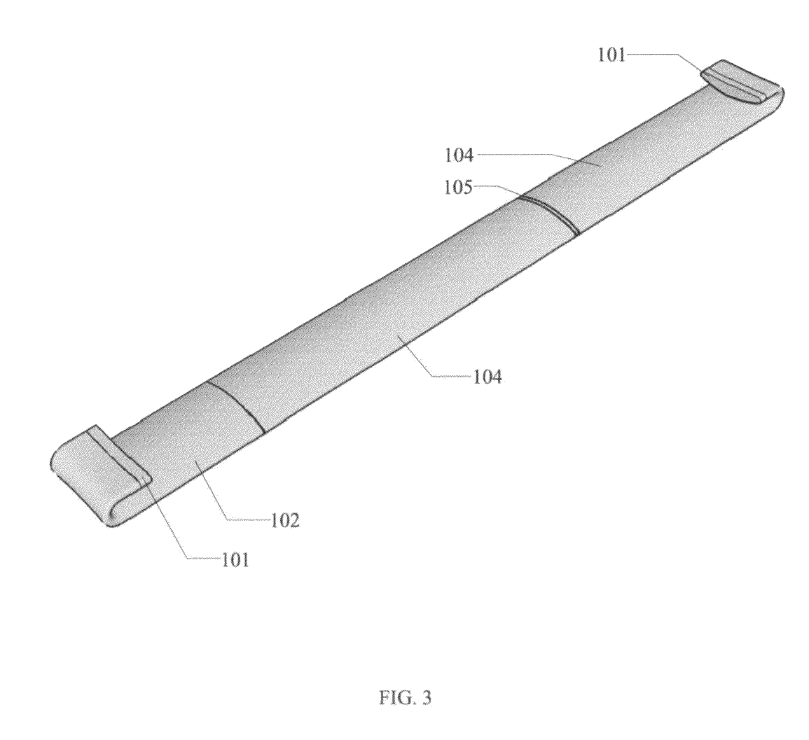

FIG. 3 shows snap on and Velcro connectors 301 and 302 for the cold wrap 100″. In the absence of connector assemblies 202 and 201, the button snap on assembly 301 snaps one end of the cold wrap 100″ to form a loop to the opposite end. Additionally, Velcro connector 302 is used to secure a loop, connecting one end to another opposite end. Both connectors 301 and 302 allow for parallel cooling activity to occur, increasing the cooling surface area for a specific area of a body by increasing the amount of body to be covered in parallel cooling. In exercise activity, cold wrap 100″ may be useful in providing cooling comfort. In such activities, the cold wrap 100″ may experience vibrations and jarring from strenuous activity. To prevent sagging, it may be necessary to provide additional support using a plastic, elastic, or rubber material.

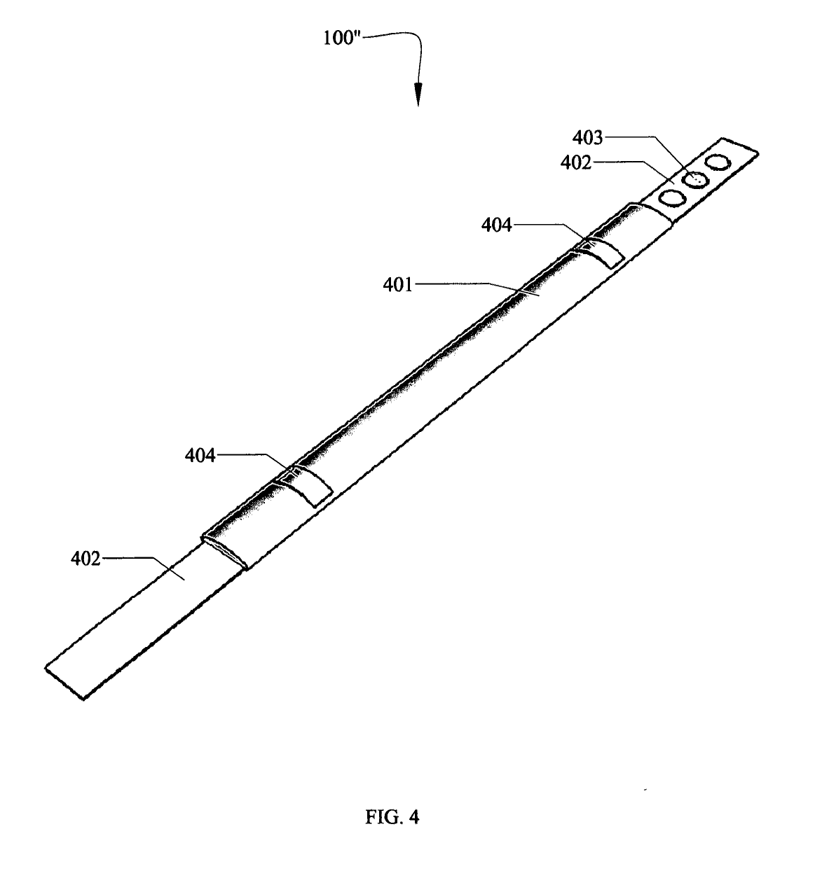

FIG. 4 shows an encasement 401 designed for enclosing the cold wrap 100, 100′, or 100″. The encasement 401 allows the cold wrap 100 to slide securely into security straps 404. Once secured, elastic bands 402 may be placed around the head of a user and secured with snaps 403. This provides a secure encasement for the cold wrap 100 in cases of activity where movement may jar the device.

FIG. 5 shows an exploded cross section perspective view of the compartmentalized cold wrap 100′. The exploded cross sectional view shows ammonium nitrate pellets 501, on the other side of the divider 105, stored within the next compartment 104. The ammonium nitrate stored in pellet form allows for faster reaction to allow a quicker temperature reduction to 36 degrees F., thus more efficient cooling.

While certain embodiments of the invention have been described, other embodiments may exist. Further, the disclosed methods' stages may be modified in any manner, including by reordering stages and/or inserting or deleting stages, without departing from the invention. While the specification includes examples, the invention's scope is indicated by the following claims. Furthermore, while the specification has been described in language specific to structural features and/or methodological acts, the claims are not limited to the features or acts described above. Rather, the specific features and acts described above are disclosed as example for embodiments of the invention.

What is claimed is:

1. A flexible cooling assembly, comprising:
an elongated cooling tube containing cooling material, the elongated cooling tube configurable to wrap around various parts of a body;
an activating mechanism associated with the cooling tube and configured to initiate at least one cooling cycle of the cooling tube; and
at least one connector configured to secure the flexible cooling assembly around at least one of the various parts of a body.

2. The cooling assembly of claim 1, wherein the cooling tube comprises a plurality of cooling compartments wherein at least one of the cooling compartments is separated from another cooling compartment by a soluble film divider and wherein dissolution of the film divider is temperature activated.

3. The cooling assembly of claim 2, wherein the soluble film divider comprises a polyvinyl alcohol water soluble film.

4. The cooling assembly of claim 1, wherein the cooling material, comprises ammonium nitrate.

5. The cooling assembly of claim 4, wherein the activating mechanism comprises a water pouch and a puncture pin configured to open the water pouch and cause water to combine with the ammonium nitrate therein initiating the at least one cooling cycle.

6. The cooling assembly of claim 1, wherein the at least one connector comprises at least one of the following:
a tube connector joint;
a tube loop configured to guide the cooling tube into multiple loops therein facilitating parallel cooling of at least one of the various body parts; and
a fastener.

7. The cooling assembly of claim 1, further comprising a flexible encasement configured to surround the cooling tube thereby providing cooling relief and minimizing jarring effects when at least one of the various body parts are in motion and wherein the various body parts include ankles, arms, legs, neck, head, and back.

8. The cooling assembly of claim 2, wherein the film divider dissolves when in contact with water approximately at or above 60 degrees Fahrenheit therein initiating a cooling cycle in the another cooling compartment of the cooling tube.

9. The cooling assembly of claim 1, wherein the cooling material comprises ammonium nitrate and wherein the at least one cooling cycle is capable of being initiated by the activating mechanism comprising a plastic pouch filled with ambient water activated by a user bursting the plastic pouch wherein when the ambient water contacts the ammonium nitrate, at least a segment of the cooling tube lowers in temperature to a temperature of approximately 36 degrees Fahrenheit.

10. The cooling assembly of claim 1, wherein at least a segment of the cooling tube is configured to remain cool once initiated at less than a temperature of 60 degrees Fahrenheit, for approximately 15 minutes.

11. The cooling assembly of claim 2, wherein at least one of the plurality of cooling compartments comprises an adjoining compartment capable of being activated upon contact with liquid from a previous cooling compartment of the plurality of cooling compartments therein causing the adjoining cooling compartment to reach a temperature of approximately 36 degrees Fahrenheit.

12. The cooling assembly of claim 3, wherein the cooling material comprises ammonium nitrate and wherein a cooling time of the cooling tube is increased by a multiplicative factor of a number of the plurality of cooling compartments within the cooling tube, thereby preventing all of the ammonium nitrate from reacting all at once by compartmenting the cooling tube therein slowing an ammonium nitrate activation rate, utilizing the poly-vinyl alcohol film as the divider from adjoining cooling compartments.

13. The cooling assembly of claim 1, whereby either end of the cooling tube friction fits into the at least one-connector, therein configured to function as a parallel cooling tube and allowing a larger surface area to cool via encircling the larger surface area multiple times.

14. The cooling assembly of claim 6, wherein the cooling tube is configured to loop into the tube loop once by friction fit therein allowing the cooling assembly to fit unto a user's head, back, or other body part where one loop is sufficient to provide relief.

15. The cooling assembly of claim 6, wherein the fastener comprises one of snap in buttons or Velcro connectors configured to serve as a secure alternative connection to tube loops therein providing adequate parallel cooling of any area of the body thru increasing cooling surface area by allowing multiple tube loops.

16. The cooling assembly of claim 2, wherein the cooling assembly distributes weight evenly therein facilitating use of the cooling assembly during heat inducing recreational or fitness activity.

17. A compartmentalized wrap around cooling apparatus, comprising:
a compartmentalized temperature activated cooling tube divided into separate cooling sections each cooling section separated by a polyvinyl alcohol water soluble film divider; and
an activating mechanism communicatively associated with the cooling tube and configured to initiate a first cooling cycle in a first section of the cooling tube;
wherein when the first cooling cycle is complete, a first film divider separating the first section and a second section of the cooling tube dissolves and initiates a second cooling cycle of the cooling tube in the second section of the cooling tube therein prolonging cooling time.

18. The cooling apparatus of claim 17, further comprising at least one of the following:
tube connector joints;
tube loops; and
fasteners;
each configured for tube looping, whereby parallel cooling occurs.
